(19) United States
(12) Patent Application Publication
(10) Pub. No.: US 2025/0099702 A1
(43) Pub. Date: Mar. 27, 2025

(54) TURBINE DRIVING MODULE AND BREATHING DEVICE

(71) Applicant: RESVENT MEDICAL TECHNOLOGY CO., LTD, Shenzhen (CN)

(72) Inventors: Kai QU, Shenzhen (CN); Rongrong ZHU, Shenzhen (CN)

(21) Appl. No.: 18/892,341

(22) Filed: Sep. 21, 2024

(30) Foreign Application Priority Data
Sep. 25, 2023 (CN) 202322604204.3

(51) Int. Cl. A61M 16/00; A61M 16/10; A61M 16/12

(52) U.S. Cl. CPC A61M 16/0066; A61M 16/125; A61M 2016/1025; A61M 2205/3375; A61M 2205/42

(57) ABSTRACT

A turbine driving module and a breathing device includes a main cavity, a turbine assembly, a first sensor group, and a second sensor group. The main cavity is divided into an oxygen mixing and noise reduction cavity, a turbine mounting cavity communicated, and a gas outlet cavity that are communicated First mounting positions having air holes communicated with the main cavity are disposed on the main cavity. A low-pressure oxygen connecting pipe, a high-pressure oxygen connecting pipe, and an air connector are communicated with the oxygen mixing and noise reduction cavity. The gas outlet pipe assembly is communicated with the gas outlet cavity. The turbine assembly is mounted in the turbine mounting cavity. Sensors of the first sensor groups are mounted on the first mounting positions. Sensors of the second sensor group are mounted in the gas outlet cavity and the oxygen mixing and noise reduction cavity.

### CROSS-REFERENCE TO RELATED APPLICATION

This application claims priority to Chinese Patent Application No. 202322604204.3, titled “TURBINE DRIVING MODULE AND BREATHING DEVICE” and filed to the China National Intellectual Property Administration on Sep. 25, 2023, the entire contents of which are incorporated herein by reference.

### TECHNICAL FIELD

The present disclosure relates to a technical field of medical devices, and in particular to a turbine driving module and a breathing device.

### BACKGROUND

A ventilator is a medical device configured to assist or maintain a patient's breathing. The ventilator assists or replaces the patient's spontaneous breathing by delivering gas (usually a mixture of air and oxygen). The ventilator commonly comprises a gas source, a gas delivery pipe, a gas heating and humidification device, a gas flow sensor, and a breathing circuit connected to the patient. Once the gas enters the breathing circuit, the gas must be delivered to the patient's lungs at an appropriate pressure and flow rate, which is generally realized by one or more fans, compressors, or air pumps. The ventilator may generate a positive pressure airflow and delivers the gas into the patient's airway to open air sacs in the lungs, allowing the oxygen to enter the blood and carbon dioxide to be discharged. At the same time, various parameters need to be detected during a gas delivery process. In the prior art, various measuring modules of the ventilator are connected through sampling ports and silicone pipes to realize a measurement function, which has a problem of accidentally falling off of the silicone pipes, increases complexity of an assembly process of the ventilator, and is costly.

### SUMMARY

In order to solve a technical problem in the prior art that pipelines connecting measuring modules of a ventilator easily come off, the present disclosure proposes a turbine driving module and a breathing device.

The present disclosure provides a turbine driving module. The turbine driving module comprises a main cavity, a low-pressure oxygen connecting pipe disposed on the main cavity, a high-pressure oxygen connecting pipe disposed on the main cavity, an air connector disposed on the main cavity, a turbine assembly, a gas outlet pipe assembly, a first sensor group, and a second sensor group.

The main cavity is divided into an oxygen mixing and noise reduction cavity, a turbine mounting cavity communicated with the oxygen mixing and noise reduction cavity, and a gas outlet cavity communicated with the turbine mounting cavity. First mounting positions are disposed on an outer side of the main cavity. The first mounting positions define air holes communicated with the main cavity.

The low-pressure oxygen connecting pipe, the high-pressure oxygen connecting pipe, and the air connector are communicated with the oxygen mixing and noise reduction cavity. The gas outlet pipe assembly is communicated with the gas outlet cavity.

The turbine assembly is mounted in the turbine mounting cavity.

Sensors of the first sensor groups are respectively mounted on the first mounting positions and respectively cover the air holes. Sensors of the second sensor group are respectively mounted in an interior of the gas outlet cavity and an interior of the oxygen mixing and noise reduction cavity.

In one optional embodiment, the first mounting positions comprise a temperature and humidity sensor mounting position and a pressure sensor mounting position. The temperature and humidity sensor mounting position is defined on the outer side of the main cavity close to the air connector. The pressure sensor mounting position is defined on an outer side surface of the gas outlet cavity of the main cavity. The first sensor group comprises a temperature and humidity sensor mounted on the temperature and humidity sensor mounting position and a pressure sensor mounted on the pressure sensor mounting position.

In one optional embodiment, the second sensor group comprises an ultrasonic oxygen concentration sensor mounted in the interior of the gas outlet cavity and a grid air damper mounted in the interior of the oxygen mixing and noise reduction cavity. The grid air damper is disposed close to a communication position of the oxygen mixing and noise reduction cavity and the turbine mounting cavity. A first signal collector connected to the grid air damper is disposed outside the main cavity.

In one optional embodiment, the main cavity comprises a housing, an upper cover plate, and a lower cover plate. The housing comprises a partition plate disposed in the housing and parallel to a bottom surface of the turbine driving module. The partition plate divides the housing into an upper cavity and a lower cavity. The partition plate defines a first communication opening to enable the upper cavity and the lower cavity to be communicated. The lower cavity is the oxygen mixing and noise reduction cavity.

The upper cavity is divided into an upper left cavity and an upper right cavity through a first vertical plate. The first vertical plate defines a second communication opening to enable the upper left cavity and the upper right cavity to be communicated. The upper left cavity is the turbine mounting cavity. The upper right cavity is the gas outlet cavity.

The lower cover plate is disposed on a bottom surface of the housing, so that a bottom portion of the oxygen mixing and noise reduction cavity is closed.

The upper cover plate is disposed on a top surface of the housing, so that a top portion of the turbine mounting cavity and a top portion of the gas outlet cavity are closed.

In one optional embodiment, partition vertical plates are disposed between the partition plate of the housing and the lower cover plate. The partition vertical plates divide the oxygen mixing and noise reduction cavity to form a mixed gas channel. The mixed gas channel is circuitous; an inlet of the mixed gas channel is communicated with the low-pressure oxygen connecting pipe and the air connector. An outlet of the mixed gas channel is communicated with the first communication opening.

In one optional embodiment, the lower cover plate comprises a groove for mounting a first sponge. A second sponge is attached to a bottom surface of the partition plate. The second sponge defines strip-shaped grooves corresponding to the partition vertical plates.

In one optional embodiment, the turbine assembly comprises a lower silicone seat, a turbine fan, and an upper silicone seat. The lower silicone seat is disposed in the turbine mounting cavity and is disposed on the partition plate. The turbine fan is mounted on the lower silicone seat, the upper silicone seat is covered on an upper portion of the turbine fan. The lower silicone seat and the upper silicone seat are enclosed to define a turbine heat dissipation cavity. The upper silicone seat defines a through hole to enable the turbine heat dissipation cavity to communicate with the turbine mounting cavity. A gas inlet of the turbine fan is located in the turbine heat dissipation cavity. A gas outlet pipe of the turbine fan is connected to the second communication opening.

In one optional embodiment, the high-pressure oxygen connecting pipe is disposed on a bottom surface of the main cavity. The low-pressure oxygen connecting pipe and the air connector are disposed side by side on one side of the main cavity.

The present disclosure further provides a breathing device. The breathing device comprises a main cavity and a turbine driving module disposed in the main cavity.

The breathing device further comprises a high-pressure oxygen module. The high-pressure oxygen module comprises a high-pressure oxygen circuit block, a high-pressure oxygen connector, a safety valve, a proportional valve, a pressure regulating valve, an air damper, a high-pressure oxygen outlet pipe, and a second signal collector. The high-pressure oxygen connector is disposed on a first end of the high-pressure oxygen circuit block. The safety valve, the proportional valve, and the pressure regulating valve are disposed on one side of the high-pressure oxygen circuit block to control an internal air path of the high-pressure oxygen circuit block. The air damper is disposed on a second end of the high-pressure oxygen circuit block. The high-pressure oxygen outlet pipe is communicated with the air damper. The second signal collector is disposed on the air damper.

Compared with the prior art, in the present disclosure, the turbine driving module of the breathing device has gas measurement functions such as temperature and humidity measurement, pressure measurement, flow measurement, oxygen concentration measurement, etc. The temperature and humidity measurement is realized by the temperature and humidity sensor mounted on an outer side of the oxygen mixing and noise reduction cavity. The pressure measurement is realized by the pressure sensor mounted on the outer side of the gas outlet cavity. The flow measurement is realized by the grid air damper mounted in a mixed gas channel of the oxygen mixing and noise reduction cavity and the first signal collector matched with the grid air damper. The oxygen concentration measurement is realized by the ultrasonic oxygen concentration sensor mounted in the gas outlet cavity. Such integrated design avoids structural splitting of the turbine driving module, reduces a risk of potential connection failure when components thereof are connected, and greatly reduces cost of the components of the turbine driving module.

A low-pressure oxygen module is integrated into the turbine driving module. In low-end models that do not require high-pressure oxygen access, the high-pressure oxygen module does not need to be configured, so there is no need to design a separate solution to adapt to the low-end models. Compared with the prior art, the cost of the turbine driving module is lower.

### BRIEF DESCRIPTION OF DRAWINGS

In order to clearly describe technical solutions in the embodiments of the present disclosure, the following will briefly introduce the drawings that need to be used in the description of the embodiments or the prior art. Apparently, the drawings in the following description are merely some of the embodiments of the present disclosure, and those skilled in the art are able to obtain other drawings according to the drawings without contributing any inventive labor.

FIG. 1 is a perspective schematic diagram of a turbine driving module according to one embodiment of the present disclosure.

FIG. 2 is an exploded schematic diagram of components in a lower cavity of the turbine driving module according to one embodiment of the present disclosure.

FIG. 3 is an exploded schematic diagram of components in an upper cavity of the turbine driving module according to one embodiment of the present disclosure.

FIG. 4 is a schematic diagram showing an air path in the turbine driving module according to one embodiment of the present disclosure when a oxygen mixing and noise reduction cavity is connected to low-pressure oxygen.

FIG. 5 is a schematic diagram showing another air path in the turbine driving module according to one embodiment of the present disclosure when the oxygen mixing and noise reduction cavity is connected to high-pressure oxygen.

FIG. 6 is a schematic diagram of the upper cavity of the turbine driving module according to one embodiment of the present disclosure where an upper cover plate and an upper silicone seat are removed.

FIG. 7 is a perspective schematic diagram of a breathing device according to one embodiment of the present disclosure.

FIG. 8 is another perspective schematic diagram of the breathing device according to one embodiment of the present disclosure.

FIG. 9 is an exploded schematic diagram of the breathing device according to one embodiment of the present disclosure.

FIG. 10 is a perspective schematic diagram of a high-pressure oxygen module according to one embodiment of the present disclosure.

FIG. 11 is an exploded schematic diagram of a high-pressure oxygen outlet pipe of the high-pressure oxygen module according to one embodiment of the present disclosure.

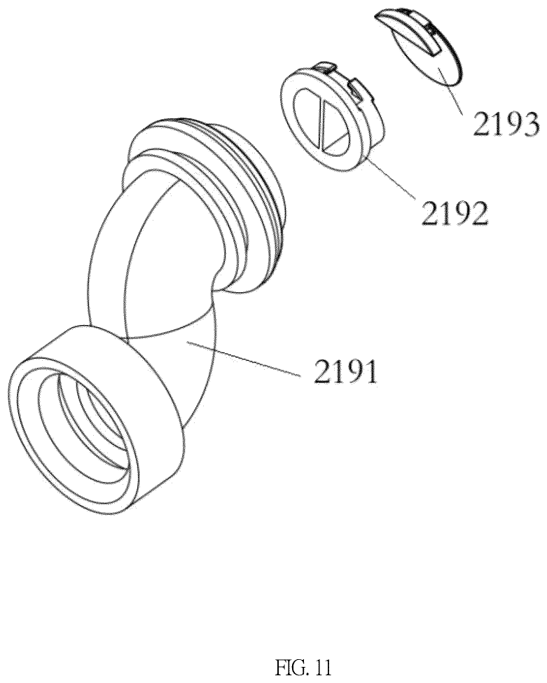

In the drawings:

1—main cavity; 2—turbine driving module; 3—high-pressure oxygen module; A—oxygen mixing and noise reduction cavity; B—turbine mounting cavity; C—gas outlet cavity; 211—housing; 212—upper cover plate; 2121—upper cover plate sealing rubber ring; 213—lower cover plate; 2131—lower cover plate sealing rubber ring; 2132—first sponge; 214—air connector; 2141—filter cotton; 215—low-pressure oxygen connecting pipe; 216—partition plate; 2161—second sponge; 2162—first communication opening; 217—first vertical plate; 2171—partition vertical plate; 2172—second communication opening; 218—second vertical plate; 219—gas outlet pipe assembly; 2191—mixed gas outlet pipe; 2192—check valve seat; 2193—check valve diaphragm; 221—temperature and humidity sensor; 2231—silicone sleeve; 224—grid air damper; 2241—first signal collector; 2211—sealing ring; 222—pressure sensor; 2221—venting silicone plug; 223—ultrasonic oxygen concentration sensor; 231—upper silicone seat; 2311—gas inlet; 232—turbine fan; 2321—gas outlet pipe; 233—lower silicone seat; 24—air hole; 31—high-pressure oxygen circuit block; 32—high-pressure oxygen connector; 33—safety valve; 34—proportional valve; 35—pressure regulating valve; 36—air damper; 37—second signal collector; 38—high-pressure oxygen outlet pipe.

### DETAILED DESCRIPTION

In order to make technical problems, technical solutions, and beneficial effects to be solved by the present disclosure clearer, the present disclosure is further described in detail below in conjunction with accompanying drawings and embodiments. It should be understood that the specific embodiments described herein are only used to explain the present disclosure and are not used to limit the present disclosure.

The principle and structure of the present disclosure are described in detail below in conjunction with the accompanying drawings and embodiments.

In the prior art, various measuring modules of a conventional breathing device are connected through sampling ports and silicone pipes to realize a measurement function, which has a problem of accidentally falling off of the silicone pipes, increases complexity of an assembly process, and is costly.

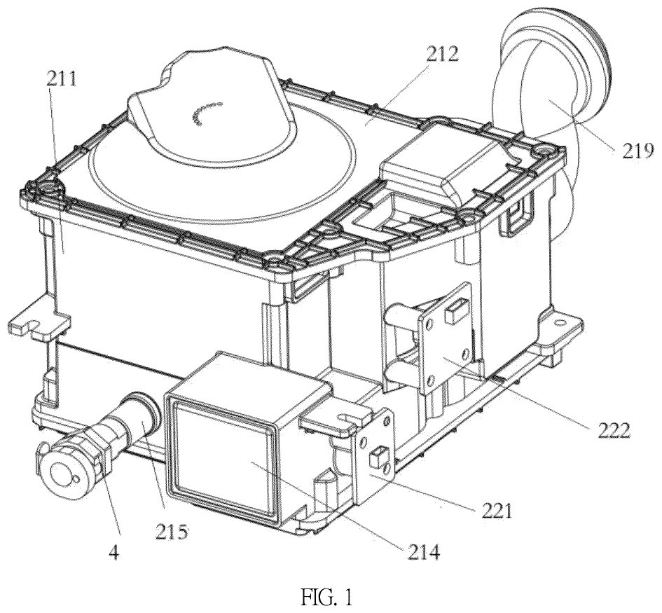

As shown in FIGS. 1-4, the present disclosure provides a turbine driving module 2. The turbine driving module 2 comprises a main cavity 1, a turbine assembly, a first sensor group, and a second sensor group. The main cavity 1 is divided into an oxygen mixing and noise reduction cavity A defined on a bottom portion of the main cavity 1, a turbine mounting cavity disposed on an upper portion of the oxygen mixing and noise reduction cavity A, and a gas outlet cavity C disposed on the upper portion of the oxygen mixing and noise reduction cavity A. The gas outlet cavity C and the turbine mounting cavity are disposed side by side. The gas outlet cavity C is communicated with the turbine mounting cavity. The oxygen mixing and noise reduction cavity A is communicated with the turbine mounting cavity. A low-pressure oxygen connecting pipe 215 and an air connector 214 are disposed side by side on one side of the main cavity 1. The high-pressure oxygen connecting pipe is disposed on a bottom surface of the main cavity 1. The low-pressure oxygen connecting pipe 215, the high-pressure oxygen connecting pipe, and the air connector 214 are communicated with the oxygen mixing and noise reduction cavity A of the main cavity 1. Oxygen entering the oxygen mixing and noise reduction cavity A from the low-pressure connecting pipe or the high-pressure oxygen connecting pipe is mixed with air entering from the air connector 214 in the oxygen mixing and noise reduction cavity A, and then is pressurized and sent to the gas outlet cavity C through the turbine assembly mounted in the turbine mounting cavity, and then sent out of the turbine driving module 2 through the gas outlet cavity C through the gas outlet pipe assembly 219. First mounting positions are defined on an outer side of the main cavity 1. The first mounting positions define air holes 24 communicated with the main cavity 1. Second mounting positions are defined in an interior of the gas outlet cavity C. Sensors of the first sensor groups are respectively mounted on the first mounting positions. Sensors of the second sensor group are respectively mounted in the interior of the gas outlet cavity C and an interior of the oxygen mixing and noise reduction cavity A.

In the present disclosure, the sensors for detection are directly disposed on one side of the main cavity 1 and are disposed inside the main cavity 1, and there is no need to connect measurement modules through silicone pipes, which reduces the complexity of a structure of the turbine driving module and a possible problem of falling off of the silicone pipes. The sensors are respectively directly fixed to mounting positions through screw holes or fastening, so that the sensors are prevented from falling off, the process is simpler, and the cost is lower.

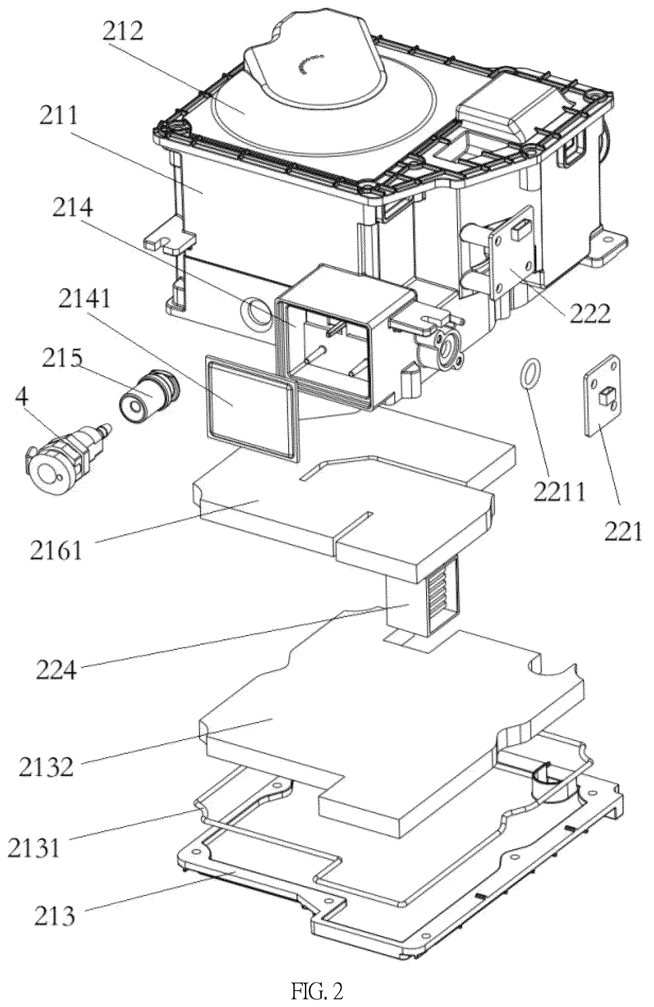

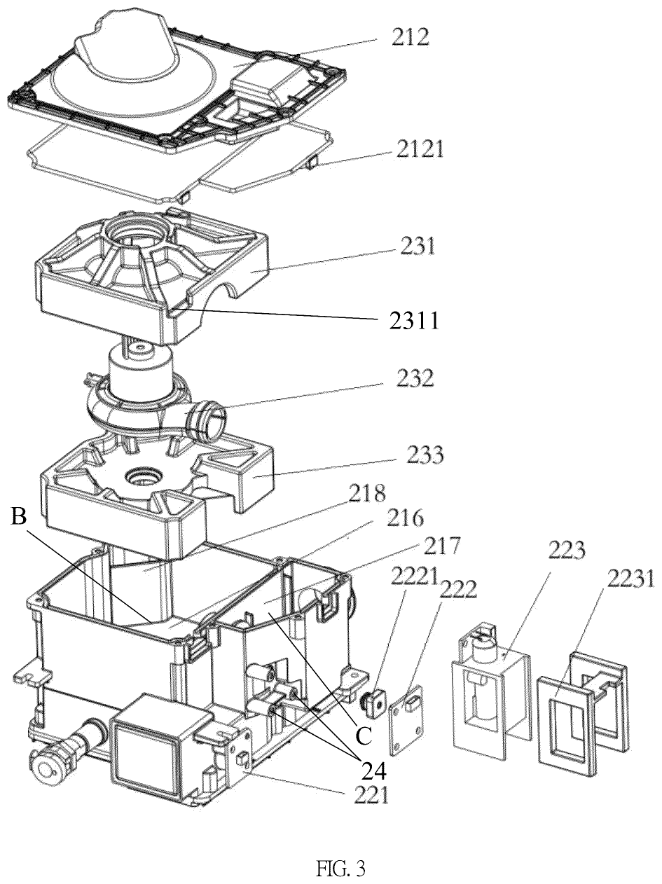

As shown in FIGS. 2 and 3, in one optional embodiment, the first sensor group comprises a temperature and humidity sensor 221 and a pressure sensor 222. The first mounting positions comprise a temperature and humidity sensor mounting position and a pressure sensor mounting position.

The temperature and humidity sensor mounting position is close to the air connector 214. The temperature and humidity sensor mounting position specifically comprises a ventilation column and first threaded columns disposed around the ventilation column. The ventilation column is cylindrical. The ventilation column is disposed on the one side of the main cavity 1 and close to the bottom portion of the main cavity 1. A first air hole is defined on a center of the ventilation column. The temperature and humidity sensor 221 is screwed with the first threaded columns and covers the first air hole of the ventilation column, and a sealing ring 2211 is disposed between the temperature and humidity sensor 221 and the ventilation column, so that the temperature and humidity sensor 221 is able to sense a temperature and a humidity of the oxygen mixing and noise reduction cavity A in the main cavity 1.

The pressure sensor mounting position is disposed on an outer side wall of the gas outlet cavity C, and the pressure sensor mounting position comprises a second air hole, a venting silicone plug 2221 mounted on the second air hole, and second threaded columns disposed around the second air hole. The pressure sensor 222 is screwed with the second threaded columns and is compressed on the venting silicone plug 2221. The pressure sensor 222 is communicated with the gas outlet cavity C to measure an air pressure on a gas outlet side of the main cavity.

The second sensor group comprises an ultrasonic oxygen concentration sensor 223 and a grid air damper 224.

A silicone sleeve 2231 is sleeved on the ultrasonic oxygen concentration sensor 223. The ultrasonic oxygen concentration sensor 223 is directly mounted in the interior of the gas outlet cavity C. The gas outlet cavity C is specifically defines a limit groove or a limit pressing structure that is able to prevent shaking of the ultrasonic oxygen concentration sensor 223, or the gas outlet cavity C comprises a cavity wall corresponding to a contour of the ultrasonic oxygen concentration sensor 223, etc., which is not specifically limited thereto, as long as the ultrasonic oxygen concentration sensor 223 is enabled to be directly mounted in the gas outlet cavity C.

The grid air damper 224 is disposed on one end of a mixed gas channel in the oxygen mixing and noise reduction cavity A. The grid air damper 224 is configured to measure an air volume. A first signal collector 2241 connected to the grid air damper 224 is disposed on the outer side of the main cavity.

In one optional embodiment, the low-pressure oxygen connecting pipe 215 is connected to the low-pressure oxygen connector 4. The gas outlet pipe assembly 219 comprises a mixed gas outlet pipe 2191, a check valve seat 2192, and a check valve diaphragm 2193 mounted on the check valve seat 2192. A first end of the gas outlet pipe is mounted on an outer surface of the main cavity at a position corresponding to the gas outlet cavity C. The first end of the gas outlet pipe is communicated with the gas outlet cavity C. The check valve seat 2192 is disposed on a second end of the gas outlet pipe to prevent gas backflow.

As shown in FIGS. 2-3, in one optional embodiment, the main cavity 1 comprises a housing 211, an upper cover plate 212, and a lower cover plate. The housing 211 is roughly a square box. The lower cover plate 213 is disposed a bottom portion of the housing 211 and seals the bottom portion of the housing 211. The housing 211 comprises a partition plate 216 disposed in a middle lower portion of the housing 211. The partition plate 216 is parallel to a bottom surface of the housing 211. The partition plate 216 divides the housing into an upper cavity and a lower cavity. The partition plate 216, a lower inner wall of the housing 211, and the lower cover plate 213 are enclosed to form the lower cavity. The lower cavity is the oxygen mixing and noise reduction cavity A. A first vertical plate 217 is disposed between inner walls of the housing 211 and is disposed on the partition plate 216. The upper cavity is divided into an upper left cavity and an upper right cavity through the first vertical plate 217. The upper left cavity is large and has a regular contour. The upper left cavity is the turbine mounting cavity. The upper right cavity is small and is the gas outlet cavity C. The upper cover plate 212 is disposed on a top surface of the turbine mounting cavity and a top surface of the gas outlet cavity C, so as to seal the turbine mounting cavity and the gas outlet cavity C, which further facilitates mounting and disassembly of the turbine assembly.

As shown in FIGS. 2 and 3, in one optional embodiment, a lower cover plate sealing rubber ring 2131 is disposed between the lower cover plate 213 and the housing 211, and an upper cover plate sealing rubber ring 2121 is disposed between the upper cover plate 212 and the housing 211, so that the upper cover plate 212, the lower cover plate 213 and the housing 211 are sealed and connected.

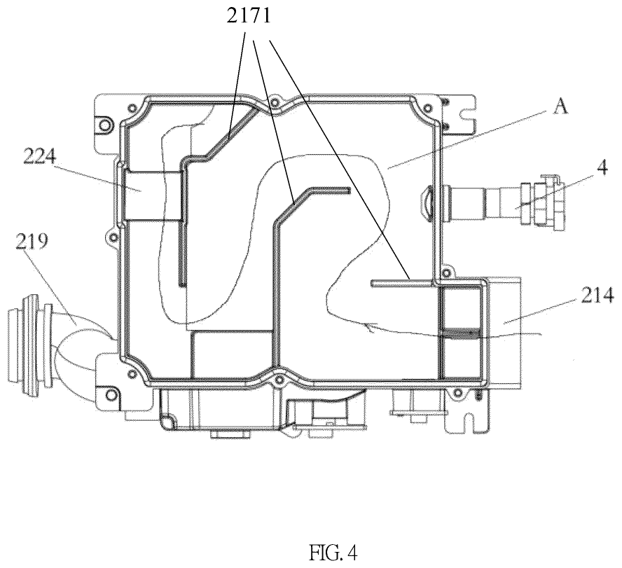

As shown in FIG. 2, partition vertical plates 2171 are disposed under the partition plate of the housing 211. The partition vertical plates 2171 extends from one side of the housing 211 toward a middle portion of the housing 211. Specifically, the partition vertical plates 2171 comprise a first partition vertical plate and a second partition vertical plate. The first partition vertical plate and the second partition vertical plate are disposed between the partition plate 216 of the housing 211 and the lower cover plate 213. The partition vertical plates 2171 divide the oxygen mixing and noise reduction cavity A to form the mixed gas channel. The mixed gas channel is circuitous. As shown in FIG. 4, the mixed gas channel is S-shaped and is in a maze form. A first end of the mixed gas channel is an inlet of the mixed gas channel. The inlet of the mixed gas channel inlet is located at the one side of the housing 211 where the low-pressure oxygen connecting pipe 215 and the air connector 214 are located. The inlet of the mixed gas channel is communicated with the low-pressure oxygen connecting pipe 215 and the air connector 214. That is, the oxygen entering the low-pressure oxygen connecting pipe 215 and the air entering the air connector 214 enter the inlet of the mixed gas channel. A second end of the mixed gas channel is an outlet of the mixed gas channel. The grille air damper 224 is disposed between the partition plate 216 of the housing 211 and the lower cover plate 213. The grille air damper 224 is disposed at a position close to the outlet of the mixed gas channel. The partition plate 216 defines a first communication opening 2162 defined on a position above the outlet of the mixed gas channel. The housing 211 further comprises a second vertical plate 218 disposed at a corner in the turbine mounting cavity B. The first communication opening 2162 of the partition plate 216 is defined below the corner of the turbine mounting cavity B. A vertical upward channel is formed between the second vertical plate 218 and two side walls at the corner of the turbine mounting cavity B of the housing 211. The vertical upward channel is configured to connect the oxygen mixing and noise reduction cavity A and the turbine mounting cavity B.

Furthermore, in one optional embodiment, the lower cover plate 213 comprises a groove for mounting a first sponge 2132. A semicircular interface disposed on the groove of the lower cover plate 213, and the semicircular interface is configured to connect the high-pressure oxygen connecting pipe. The first sponge 2132 defines a notch corresponding to the grid air damper 224, and a semicircular notch corresponding to the semicircular interface. A second sponge 2161 is attached to a bottom surface of the partition plate 216. The second sponge 2161 defines strip-shaped grooves corresponding to the partition vertical plates 2171.

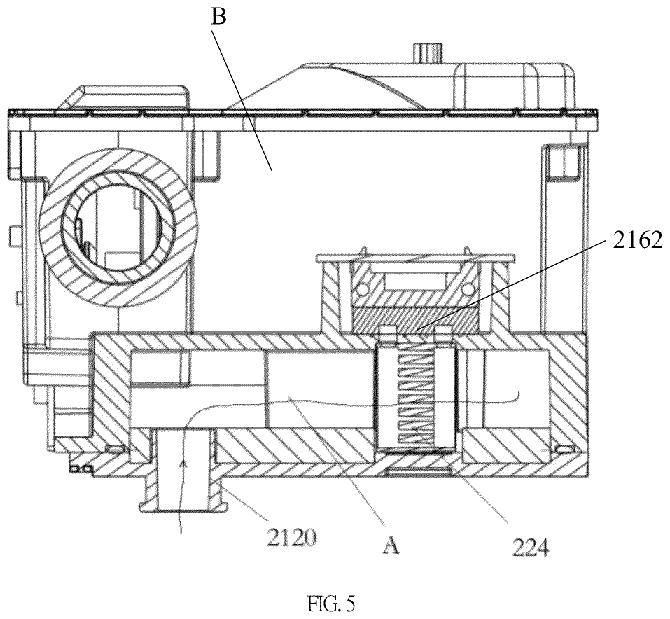

As shown in FIG. 5, the high-pressure oxygen connecting pipe 2120 is connected to the lower cover plate 213 and is close to the grid air damper 36. A portion of the mixed gas channel between the high-pressure oxygen connecting pipe 2120 and the grid air damper 36 is shorter than a portion of the mixed gas channel between the low-pressure oxygen connecting pipe 215 and the grid air damper 36.

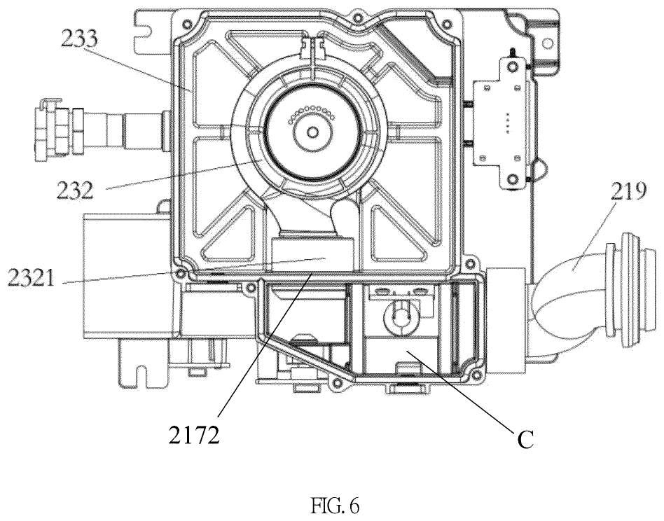

As shown in FIGS. 3 and 6, the turbine assembly is mounted in the turbine mounting cavity B. The turbine assembly comprises a lower silicone seat 233, a turbine fan 232, and an upper silicone seat 231. The turbine fan 232 is mounted between the upper silicone seat 231 and the lower silicone seat 233. A turbine heat dissipation cavity is formed between the upper silicone seat 231 and the lower silicone seat 233. An gas inlet 2311 is defined on a corner of the upper silicone seat 231, and the gas inlet 2311 is communicated with the vertical upward channel disposed at the corner of the turbine mounting cavity B, so that the gas mixed by the oxygen mixing and noise reduction cavity A flows upward into the turbine mounting cavity B through the vertical upward channel, and then enters the turbine heat dissipation cavity between the upper silicone seat 231 and the lower silicone seat 233 through the gas inlet 2311 to dissipate heat for the turbine fan 232. The gas is then sucked in from an air intake of the turbine fan 232. The gas outlet pipe 2321 of the turbine fan 232 (the upper silicone seat 231 and the lower silicone seat 233 define of the gas outlet pipe 2321) is connected to the first vertical plate 217. A second communication opening 2172 is defined on the first vertical plate 217, so that the gas pressurized by the turbine fan 237 enters the gas outlet cavity C, and then flows out from the gas outlet pipe assembly 219 through the gas outlet cavity C after being detected by the sensors.

In a working state, the oxygen mixing and noise reduction cavity A is in a negative pressure state, and a path of the air entering the oxygen mixing and noise reduction cavity A is shown in FIG. 4. When the air enters the oxygen mixing and noise reduction cavity A, the air first passes through a filter cotton 2141. The filter cotton 2141 filters impurities in the air, and the temperature and humidity sensor 221 mounted on the side wall of the housing measures the temperature and humidity of the filtered air entering the oxygen mixing and noise reduction cavity A. When low-pressure oxygen is used, the low-pressure oxygen is transported to the oxygen mixing and noise reduction cavity A through the low-pressure oxygen connector and the low-pressure oxygen connecting pipe. The filtered air and the low-pressure oxygen are mixed in the “S-shaped” mixed gas channel of the oxygen mixing and noise reduction cavity A, and the mixed gas forms pressure difference when passing through the grid air damper 36 mounted in the oxygen mixing and noise reduction cavity A. A flow rate passing through the mixed gas channel is obtained after calculation by the first signal collector 2241. Then, the mixed gas enters the turbine heat dissipation cavity through a channel between the oxygen mixing and noise reduction cavity A and the turbine heat dissipation cavity.

When high-pressure oxygen is used, the high-pressure oxygen is output from the high-pressure oxygen outlet pipe 38 of the high-pressure oxygen module 3 and enters the oxygen mixing and noise reduction cavity A through a high-pressure oxygen inlet of the turbine driving module 2. As shown in FIG., the high-pressure oxygen is mixed with the filtered air and passes through the grid air damper 36 to measure the flow rate, and then enters the turbine heat dissipation cavity through the channel between the oxygen mixing and noise reduction cavity A and the turbine heat dissipation cavity.

The turbine driving module 2 has gas measurement functions such as temperature and humidity measurement, pressure measurement, flow measurement, oxygen concentration measurement, etc. The temperature and humidity measurement is realized by the temperature and humidity sensor 221 mounted on the outer side of the oxygen mixing and noise reduction cavity A. The pressure measurement is realized by the pressure sensor 222 mounted on the outer side of the gas outlet cavity C. The flow measurement is realized by the grid air damper mounted in a mixed gas channel of the oxygen mixing and noise reduction cavity A and the first signal collector 2241 cooperate with the grid air damper. The oxygen concentration measurement is realized by the ultrasonic oxygen concentration sensor 223 mounted in the gas outlet cavity C. Such integrated design avoids structural splitting of the turbine driving module 2, reduces a risk of potential connection failure when components thereof are connected, and greatly reduces cost of the components of the turbine driving module 2.

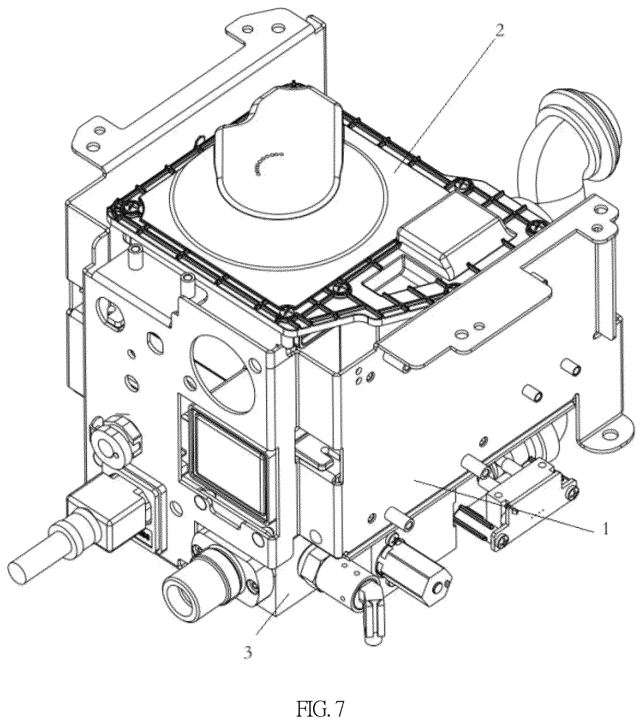

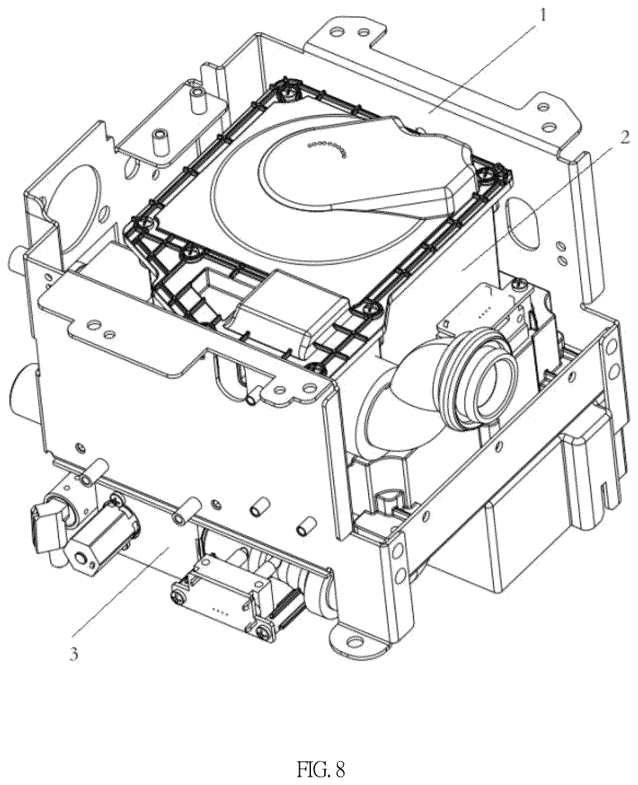

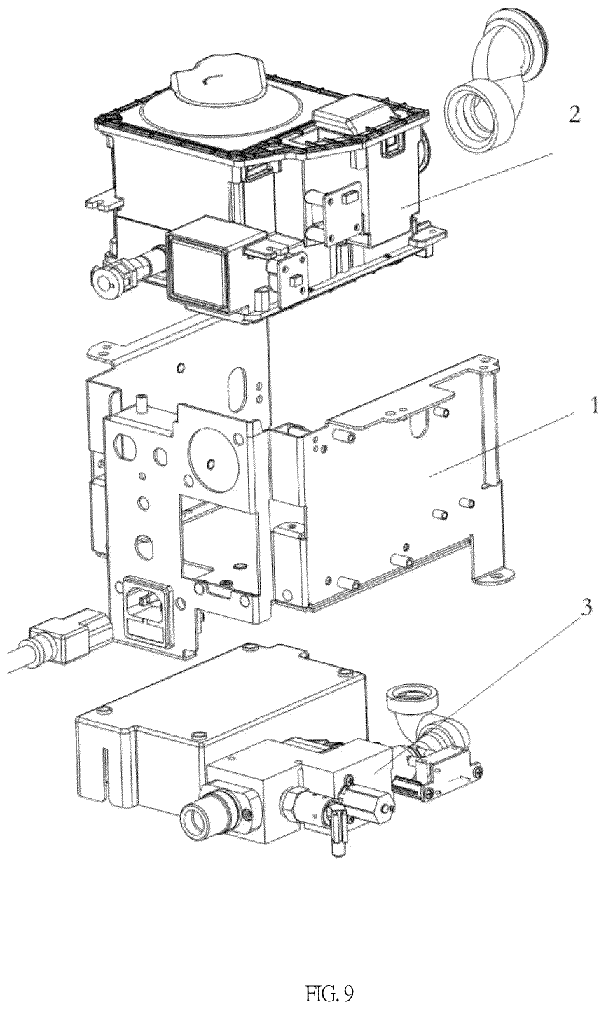

As shown in FIGS. 7-9, the present disclosure further provides a breathing device. The breathing device comprises a main cavity 1 and the turbine driving module 2 mentioned above.

In one optional embodiment, the main cavity 1 comprises the high-pressure oxygen module mounting position disposed below the turbine driving module 2. When the high-pressure oxygen is not needed, the high-pressure oxygen module 3 is omitted. When the high-pressure oxygen is needed, the high-pressure oxygen module 3 is directly mounted in the main cavity 1 of the breathing device and connected to the turbine driving module 2.

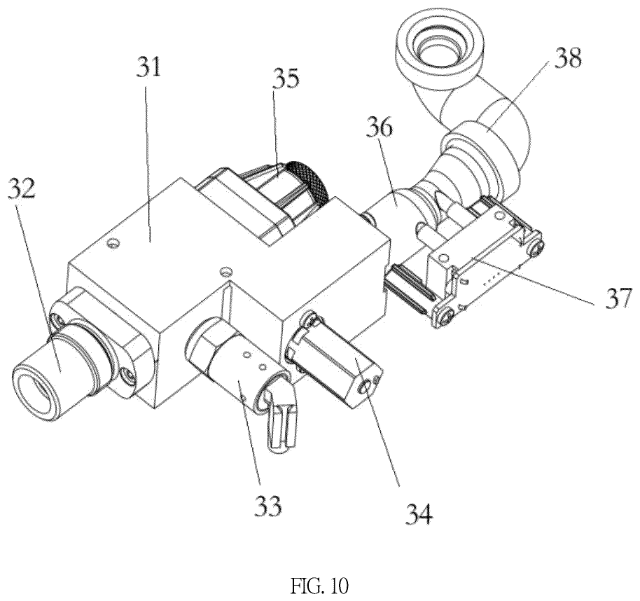

As shown in FIG. 10, the high-pressure oxygen module 3 comprises a high-pressure oxygen circuit block 31, a high-pressure oxygen connector 32, a safety valve 33, a proportional valve 34, a pressure regulating valve 35, an air damper 36, a high-pressure oxygen outlet pipe 38, and a second signal collector 37. A first end of the high-pressure oxygen circuit block 31 is an inlet of the high-pressure oxygen, and a second end of the high-pressure oxygen circuit block is an outlet of the high-pressure oxygen. The high-pressure oxygen connector 32 is disposed on the first end of the high-pressure oxygen circuit block 31. The safety valve 33, the proportional valve 34, and the pressure regulating valve 35 are disposed on one side of the high-pressure oxygen circuit block 31 to control an internal air path of the high-pressure oxygen circuit block. The air damper 36 is disposed on the second end of the high-pressure oxygen circuit block 31. The high-pressure oxygen outlet pipe 38 is communicated with the air damper 36. The second signal collector 37 is disposed on the air damper 36.

It should be noted that the terms used in the present disclosure are for a purpose of describing particular embodiments only and does not limit the present disclosure. As used in the present disclosure, singular forms are intended to comprise the plural forms as well, unless the context clearly dictates otherwise. It is also understood that the term “and/or” as used herein refers to and comprises features, steps, operations, devices, components and/or combinations thereof.

Unless specifically stated otherwise, relative arrangements of components, steps, numerical expressions, and numerical values set forth in these embodiments do not limit the scope of the present disclosure. In addition, it should be understood that, for ease of description, the sizes of the parts shown in the accompanying drawings are not drawn according to actual proportional relationships. Techniques, methods, and apparatus known to those skilled in the art may not be discussed in detail, but where appropriate, the techniques, methods, and apparatus should be considered as part of the authorization specification. In all embodiments shown and discussed herein, any specific value should be construed as exemplary only and not as a limitation. Thus, other examples of the exemplary embodiments may have different values. It should be noted that similar reference numerals and letters refer to similar items in the following drawings, and therefore, once an item is defined in one figure, it does not need to be further discussed in the subsequent figures.

It should be understood in the description of the present disclosure that directional terms such as “front”, “rear”, “upper”, “lower”, “left”, “right”, “vertical”, “horizontal”, “perpendicularly”, “top”, “bottom”, etc. indicate direction or position relationships shown based on the drawings, and are only intended to facilitate the description of the present disclosure and the simplification of the description rather than to indicate or imply that the indicated device or element must have a specific direction or constructed and operated in a specific direction, and therefore, shall not be understood as a limitation to the present disclosure. The directional terms “inside” and “outside” refer to the inside and outside relative to the outline of each component itself.

For ease of description, spatially relative terms, such as “above”, “on”, “on a surface”, “over”, etc., may be used to describe spatial positional relationships between one device or feature and other devices or features as shown in the figures. It should be understood that the spatially relative terms are intended to encompass different orientations in use or operation in addition to the orientation depicted in the figures. For example, if a device in the accompanying drawings is inverted, another device described as “above” or “on” the device is positioned as under the device or below the device. Thus, the exemplary term “above the device” may comprise two orientations such as above the device or below the device. The device may also be positioned (rotated 90 degrees or at other orientations) in other different ways, and the spatially relative description used herein is interpreted accordingly.

In addition, it should be noted that the terms such as “first” and “second” are used to limit the parts, and are merely to distinguish corresponding parts. Unless otherwise stated, the above terms have no special meaning and therefore cannot be understood as limiting the protection scope of the present disclosure.

The above description is only optional embodiments of the present disclosure and is not intended to limit the present disclosure. For those skilled in the art, the present disclosure may have various modifications and variations. Any modification, equivalent replacement, improvement, etc. made within the spirit and principle of the present disclosure shall be comprised in the protection scope of the present disclosure.

What is claimed is:

1. A turbine driving module, comprising:
a main cavity,
a low-pressure oxygen connecting pipe disposed on the main cavity,
a high-pressure oxygen connecting pipe disposed on the main cavity,
an air connector disposed on the main cavity,
a turbine assembly,
a gas outlet pipe assembly,
a first sensor group, and
a second sensor group;
wherein the main cavity is divided into an oxygen mixing and noise reduction cavity, a turbine mounting cavity communicated with the oxygen mixing and noise reduction cavity, and a gas outlet cavity communicated with the turbine mounting cavity; first mounting positions are disposed on an outer side of the main cavity, the first mounting positions define air holes communicated with the main cavity;
wherein the low-pressure oxygen connecting pipe, the high-pressure oxygen connecting pipe, and the air connector are communicated with the oxygen mixing and noise reduction cavity; the gas outlet pipe assembly is communicated with the gas outlet cavity;
wherein the turbine assembly is mounted in the turbine mounting cavity;
wherein sensors of the first sensor groups are respectively mounted on the first mounting positions and respectively cover the air holes; sensors of the second sensor group are respectively mounted in an interior of the gas outlet cavity and an interior of the oxygen mixing and noise reduction cavity.

2. The turbine driving module according to claim 1, wherein the first mounting positions comprise a temperature and humidity sensor mounting position and a pressure sensor mounting position; the temperature and humidity sensor mounting position is defined on the outer side of the main cavity close to the air connector; the pressure sensor mounting position is defined on an outer side surface of the gas outlet cavity of the main cavity; the first sensor group comprises a temperature and humidity sensor mounted on the temperature and humidity sensor mounting position and a pressure sensor mounted on the pressure sensor mounting position.

3. The turbine driving module according to claim 1, wherein the second sensor group comprises an ultrasonic oxygen concentration sensor mounted in the interior of the gas outlet cavity and a grid air damper mounted in the interior of the oxygen mixing and noise reduction cavity; the grid air damper is disposed close to a communication position of the oxygen mixing and noise reduction cavity and the turbine mounting cavity; a first signal collector connected to the grid air damper is disposed outside the main cavity.

4. The turbine driving module according to claim 1, wherein the main cavity comprises a housing, an upper cover plate, and a lower cover plate; the housing comprises a partition plate disposed in the housing and parallel to a bottom surface of the housing; the partition plate divides the housing into an upper cavity and a lower cavity; the partition plate defines a first communication opening to enable the upper cavity and the lower cavity to be communicated; the lower cavity is the oxygen mixing and noise reduction cavity;
wherein the upper cavity is divided into an upper left cavity and an upper right cavity through a first vertical plate; the first vertical plate defines a second communication opening to enable the upper left cavity and the upper right cavity to be communicated; the upper left cavity is the turbine mounting cavity; the upper right cavity is the gas outlet cavity;
wherein the lower cover plate is disposed on a bottom surface of the housing, so that a bottom portion of the oxygen mixing and noise reduction cavity is closed;
wherein the upper cover plate is disposed on a top surface of the housing, so that a top portion of the turbine mounting cavity and a top portion of the gas outlet cavity are closed.

5. The turbine driving module according to claim 4, wherein partition vertical plates are disposed between the partition plate of the housing and the lower cover plate; the partition vertical plates divide the oxygen mixing and noise reduction cavity to form a mixed gas channel; the mixed gas channel is circuitous; an inlet of the mixed gas channel is communicated with the low-pressure oxygen connecting pipe and the air connector; an outlet of the mixed gas channel is communicated with the first communication opening.

6. The turbine driving module according to claim 5, wherein the lower cover plate comprises a groove for mounting a first sponge; a second sponge is attached to a bottom surface of the partition plate; the second sponge defines strip-shaped grooves corresponding to the partition vertical plates.

7. The turbine driving module according to claim 4, wherein the turbine assembly comprises a lower silicone seat, a turbine fan, and an upper silicone seat; the lower silicone seat is disposed in the turbine mounting cavity and is disposed on the partition plate; the turbine fan is mounted on the lower silicone seat; the upper silicone seat is covered on an upper portion of the turbine fan; the lower silicone seat and the upper silicone seat are enclosed to define a turbine heat dissipation cavity; the upper silicone seat defines a through hole to enable the turbine heat dissipation cavity to be communicated with the turbine mounting cavity; a gas inlet of the turbine fan is located in the turbine heat dissipation cavity; a gas outlet pipe of the turbine fan is connected to the second communication opening.

8. The turbine driving module according to claim 1, wherein the high-pressure oxygen connecting pipe is disposed on a bottom surface of the main cavity; the low-pressure oxygen connecting pipe and the air connector are disposed side by side on one side of the main cavity.

9. A breathing device, comprising:
a main cavity, and
a turbine driving module according to claim 1.

10. The breathing device according to claim 9, wherein the breathing device further comprises a high-pressure oxygen module; the high-pressure oxygen module comprises a high-pressure oxygen circuit block, a high-pressure oxygen connector, a safety valve, a proportional valve, a pressure regulating valve, an air damper, a high-pressure oxygen outlet pipe, and a second signal collector; the high-pressure oxygen connector is disposed on a first end of the high-pressure oxygen circuit block; the safety valve, the proportional valve, and the pressure regulating valve are disposed on one side of the high-pressure oxygen circuit block to control an internal air path of the high-pressure oxygen circuit block; the air damper is disposed on a second end of the high-pressure oxygen circuit block; the high-pressure oxygen outlet pipe is communicated with the air damper; the second signal collector is disposed on the air damper.
